(19) United States
(12) Patent Application Publication
(10) Pub. No.: US 2015/0257970 A1
(43) Pub. Date: Sep. 17, 2015

(54) DEVICE AND METHOD FOR REDUCING PAIN

(71) Applicants: Martin Mücke, Bonn (DE); Tobias Weigl, Bonn (DE); Johannes Weigl, Dusseldorf (DE)

(72) Inventors: Martin Mücke, Bonn (DE); Tobias Weigl, Bonn (DE); Johannes Weigl, Dusseldorf (DE)

(21) Appl. No.: 13/985,241

(22) PCT Filed: Feb. 17, 2012
(86) PCT No.: PCT/EP12/00706

§ 371 Date: Jan. 23, 2014

(30) Foreign Application Priority Data
Feb. 17, 2011 (DE) 10-2011-011-610.9
Dec. 5, 2011 (DE) 10-2011-120-068.5

(51) Int. Cl. A61H 23/02; A61H 23/00; A61B 5/15

(52) U.S. Cl. CPC A61H 23/02; A61B 5/150137; A61B 5/150106; A61H 23/00; A61H 2201/10

(57) ABSTRACT

The invention relates to a device and a method for reducing pain on a human or animal body using at least two electrodes as part of an electrode assembly. The device includes an electrode assembly, the surface of which can be brought into contact with the surface of a human or animal body, a pulse generating unit for applying current to the electrode assembly, and a vibration unit for vibration of the electrode assembly.

### REFERENCE TO RELATED APPLICATIONS

This application is the national stage application under 35 U.S.C. §371 of the International Application No. PCT/EP2012/000706 filed Feb. 17, 2012, which claims the benefit of German Patent Application No. 102011120068.5, filed Dec. 5, 2011, and German Patent Application No. 102011011610.9, filed Feb. 17, 2011, the entire disclosures of which are incorporated herein by reference in their entireties for all purposes.

### FIELD OF THE INVENTION

The invention relates to a device for reducing pain in medical procedures on a human or animal body part, said device having a vessel through which a fluid flows, in particular blood.

### BACKGROUND OF THE INVENTION

In medical diagnostics, millions of vaccinations, infusions, venous punctures, transfusions and blood samples are performed and taken each day. These medical procedures on a human or animal body must be performed rapidly because of the large number of procedures, if for no other reason.

Within the context of venous blood sampling, blood in the blood vessel is brought to stasis by means of a venous occlusion device, also known as a tourniquet. The skin is then disinfected over the puncture site using an alcoholic disinfectant. The blood vessel is then punctured through the skin using a sterile cannula, so that the blood can be collected in collecting devices suitable for this purpose, such as blood collection tubes. The tourniquet is released during or after successful blood collection, the cannula is removed and the puncture site is provided with a wound dressing under pressure. The same principle (arterial or venous) is used in the principle of the infusion, except that a solution is administered.

The preparatory measures and the pain caused by the needle puncture are often associated with a stress reaction (distress). In children in particular, unpredictable severe pain may occur with minimally invasive medical procedures, for example, venous punctures, intramuscular injections or when administering infusions. These procedures may therefore cause anxiety and stress reactions in both the child and the parent, which can result in an increased perception of pain on the one hand, and problems in performing the procedure on the other hand. According to one study by Broome et al. (1990), invasive medical procedures are among the most feared events in childhood. This experience of pain is very often reinforced and therefore it is not uncommon for adults to have an elevated level of anxiety about minimally invasive procedures. Since pain is a personal experience which some people perceive more strongly and others more weakly, this pain may even cause some people to react to this experience of pain with an anxiety bordering on panic.

In practice, various methods are known which may reduce pain. In addition to various topical anesthetics administered by injection, transdermal topical anesthesia is frequently used. Furthermore, iontophoresis, use of an ice spray and psychotherapy are all suitable methods of reducing pain. All these methods and processes are complex in terms of resources because they require time and personnel and accordingly, are cost-intensive. Furthermore, transcutaneous electrical stimulation (TENS) is also known for pain reduction.

US 2005/0038463 A1 describes the use of a device for sampling blood from a fingertip for blood sugar determination in which TENS is used to suppress or reduce the pain stimulus caused by the puncture. This device includes a stabilization block, which may typically be made of metal, stainless steel or plastic. The stabilization block also includes a finger opening with an opening diameter of 1.5 to 3 cm, which, in a preferred exemplary embodiment, may be designed with a trough shape and may have a length of 1-11 cm. At least two electrodes, which are in contact with a finger placed in the finger opening, may be applied to the inside of the finger opening. These electrodes are made of an electrically conductive material. In a preferred embodiment, the finger is compressed on insertion into the finger opening. Administration of the stimulus current for the TENS via the electrodes may take place here through an integrated circuit or an external TENS control unit. The stimulus currents applied here amount to at least 50 mA, their intensity and frequency being adaptable by a regulator. Good pain reduction is typically achieved with stimulus currents of 5-70 mA with a frequency of 50-100 Hz and a pulse width of 50-200 μs. However, this method does not make it possible to sample larger volumes of blood such as those often needed in clinical diagnostic procedures. Furthermore, the device described in US 2005/0038463 does not allow therapeutic administration of vaccines or infusions, for example.

US 2004/0015188 A1 describes a device for reducing pain that may occur with superficial therapeutic injections or in collecting a specimen from superficial body tissue. This device consists of a battery arrangement, an ON button, an LED display, an injection needle and a holder for an injection syringe, and a horseshoe-shaped clamping body which holds the electrodes. The injection syringe may be secured by the holder and the clamping body comprising the electrodes on the battery arrangement in a contact closure. Other embodiments may be designed like a glove, in which the electrode surfaces may be applied to the palm and fingers or the electrodes may be applied directly to the TENS generator. However, the embodiments described here do not include a tourniquet for reducing the venous blood flow, which is why they have only limited suitability for collecting blood.

US 2005/0165459 A1 describes the design of a TENS control unit and an electrode connected thereto, which can be used for suppressing pain with injections. In a preferred embodiment, the electrode may consist of concentric circular pieces arranged around a puncture site. The individual circular electrode elements connected to one another in a nonconducting manner can be controlled by the TENS control unit in such a way that a stimulus current for pain suppression can be generated after applying the device to the skin around the puncture site. US 2005/0165459 A1 also discloses a possible programming of the control unit for this purpose, so that a stimulus current can be applied to the electrode components designed in this way.

WO 99/56631 A1 describes a specimen collection system for pain stimulus reduction in collecting a bone marrow specimen in which a biopsy needle is connected to a TENS control unit in an electrically conducting manner and another electrode is attached to the patient's skin. This makes it possible to apply the stimulus current directly to the puncture site of the bone to achieve the best possible analgesic effect.

WO 2008/129555 describes a device for transcutaneous electrical stimulation for pain reduction, in which stimulus currents that allow effective pain reduction are used. The stimulus currents used here may have a triangular or delta shape with a variable pulse period and amplitude. In a preferred embodiment, the stimulus current is applied over skin areas of 16 mm², which are preferably located 4 cm apart from one another with a stimulus current of 100 mA. The stimulus current pulses here have a variable amplitude whose maximum is approx. 100 mA. In another preferred embodiment, the pulse width is 25-50 μS, with the rise time and fall time not amounting to more than 5% of the pulse width, with an interpulse interval of 0.1 to 3 ms. The amplitude of the pulses is also modulated, so that the respective pulse amplitude is preferably 70-100% of the preset pulse amplitude.

### SUMMARY

Against this background, the object of the invention is to provide an improved device for reducing pain that may occur in a human or animal body part. In particular the invention should make it possible to reduce the pain associated with injections, infusions, transfusions, blood sampling or venous punctures.

If the device according to the invention is used with a medical procedure on a human or animal body part having a vessel through which a fluid flows, then a tourniquet may be provided with a device according to the invention, said tourniquet being suitable for application to the body part and then being able to reduce the flow of fluid when it is applied to the body part. Furthermore, the device according to the invention has as least two electrodes and a current source connected to the electrodes. The pain threshold with the device according to the invention is presumably raised by electrical stimulation. The basis of this principle is the so-called gate control hypothesis, according to which endogenous inhibitory mechanisms for the pain fibers in the spinal cord are activated by stimulating afferent fast conducting Aβ fibers. Secondly, descending inhibitory nerve paths are stimulated to increase the release of endorphins. Studies have shown that free nerve endings of the slightly myelinized Aδ and C fibers function as the most important skin nociceptors (Melzack, R., Wall, P. D., Pain mechanisms: A new theory, Science, 1965, 150: 971-979).

These skin nociceptors are excited by harmful stimuli such as puncturing with a cannulated needle to collect blood through the skin and vascular tissue (intima, media, adventitia), for example, and they cause the perception of pain.

For the purpose of this invention, a vessel is understood to be any flow-through space in a body part through which a fluid flows, the flow of which is to be reduced for a medical procedure. A vein in this sense is also a vessel. The fluid is understood in particular to be blood, lymph or cerebrospinal fluid. For the sake of simplicity, the invention is described in greater detail below on the basis of a vein and the blood flow, although the invention is not limited to this embodiment.

The device according to the invention has a tourniquet, which is suitable for application to a body part and can reduce the flow of fluid when it is attached to the human or animal body part. The invention can be easily implemented with the tourniquets known in practice, which works together with at least two electrodes and a current source connected to the electrodes for implementation of the invention. Within the device according to the invention, the tourniquet is provided primarily to reduce the venous blood flow. To do so, the tourniquet according to the invention should be suitable for application to the body part. In particular the tourniquet should be applied to the body part in such a way that it need not be held in place by the user, i.e., it is held independently on the body part. This is achieved in particular when the tourniquet completely encloses the body part and is secured on the body part by internal tension and/or by creating a pressure, so that a reduction in venous blood flow can be achieved by this creation of pressure. However, it is not absolutely necessary for the tourniquet to surround the body part in order to fulfill its functions within the scope of the invention. It is thus conceivable that an object attached to the body part by an adhesive is used without completely surrounding the body part, and the pressure achieved through the inherent tension is applied to the vein in the body part to thereby reduce the venous blood flow. Other examples of devices that may be used to create stasis in the blood flow within the scope of the present invention include, for example, products made of fibers, in particular preferably textiles that exert a compression effect, such as those known as compression stockings. Such a material may be used to produce a covering for the entire body part, said covering then being suitable for application to the body part (it can be pulled over the body part) and being suitable for reducing the venous blood flow due to the compression of the body part.

It is also possible to use as the tourniquet an object, which is attached to the body part without completely surrounding it, and which exerts a pressure on the vein in the body part by antistatic adhesion, for example, in order to thereby reduce the venous blood flow, for example.

The device according to the invention has at least two electrodes and one current source connected to the electrodes. The electrodes and the current source may be designed as components that are separate from the tourniquet, so that the device according to the invention includes several components, optionally independent of one another. In a preferred embodiment, however, the electrodes and/or the current source are connected to the tourniquet and can be applied to the body part by means of the tourniquet. This makes it possible for the device according to the invention to be easy to handle as such. Furthermore, this achieves the result that all the components of the device according to the invention remain together and cannot be lost. Finally, this preferred embodiment of the device achieves the result that the user need not hold any of the components of the device according to the invention. If all the components of the device according to the invention are applied to the body part by means of the tourniquet, then the user has his hands free to perform the medical procedure.

In particular, the electrodes are preferably designed so that a transcutaneous electrical stimulation for pain reduction can be achieved with them.

The electrodes are designed to work together with the body part, in particular to introduce a current into the body part. To this end, the electrodes may be designed so that they can be attached to the body part, for example, being adhered to the body part. However, the electrodes are preferably designed as part of the tourniquet and are arranged in or on the tourniquet in such a way that a separate handling of the electrodes is not necessary. In a preferred embodiment, the electrodes and the tourniquet are designed so that merely attaching the tourniquet to the body part brings the electrodes into a position in which they can manifest their effect.

In a preferred embodiment, the tourniquet has an intervention site. For example in the case of tourniquet formed as a coating of a woven fiber fabric that applies compression, such an intervention site may be implemented in that an opening is provided in the fabric as an intervention site for the medical procedure. In such a preferred embodiment, the electrodes are placed on the tourniquet, namely on both sides of the intervention site, one electrode on one edge of the intervention site and another electrode preferably opposite this edge on another edge of the intervention site. Such an embodiment of the device according to the invention simplifies the handling of the device. First, it is possible to ensure through the intervention site and its arrangement in the tourniquet that the tourniquets are always applied so that the position in relation to the location where the intervention is to be performed corresponds to a desired location. Furthermore, by applying the electrodes to the tourniquet and on both sides of the intervention site it is possible to achieve a rapid and simple placement of the electrodes. In such a preferred embodiment, the electrodes are arranged and embodied in such a way that transcutaneous electrical stimulation for pain reduction can be performed. It is especially preferable to apply the electrodes laterally and medially in relation to the longitudinal axis of the body part, on both sides of the intervention site in the tourniquet. This facilitates the development of a stimulus current field which can be utilized to suppress pain at the intervention site.

In another preferred embodiment, the electrodes each have an area of at least 1200 mm², in particular preferably at least 1600 mm² and most especially preferably at least 1800 mm². Due to this embodiment of the electrodes according to the invention, a stimulus current field of a sufficient size develops, so that the pain stimuli can be effectively suppressed. The area is preferably embodied as circular or elliptical.

In another preferred embodiment, the electrodes here are connected via electric conductors to a control unit through which the electrodes can be controlled. According to the invention, electrical conductors which are connected to the electrodes via plug contacts, for example, may be used for this purpose, as is known in the state of the art. Known embodiments of TENS units from the state of the art may be used to control the electrodes, such as those known from U.S. Pat. No. 5,350,414.

In another preferred embodiment, the control unit communicates wirelessly with the controllable electrodes, so that the stimulus current can be controlled without the control unit being electrically connected to the electrodes. This preferred embodiment makes it possible for the control unit to be placed at a spatial distance from the electrodes. This has the advantage that the freedom of movement and the access to the intervention site on the patient are not limited by electric conductors. In addition, this also permits facilitated operation of Bluetooth® or other conventional transmitter-receiver combinations can be chosen here as possible embodiments of wireless communication.

In another preferred embodiment, the control unit which communicates with the electrodes in a hardwired or wireless method generates or controls a stimulus current from one electrode to another electrode, the current being monophasic or biphasic.

In another preferred embodiment, the amplitude and/or frequency of the monophasic or biphasic stimulus current can be regulated by the control unit. Stimulus current frequencies of 1-200 Hz are especially preferred, more preferably 100-160 Hz or 25-50 Hz. This permits the stimulus current to be adjusted to the individual needs of the patient to thereby achieve an optimal pain reduction.

In another preferred embodiment, the device according to the invention has two sections that can be connected by a closure. By connecting these sections, an electric circuit provided in the tourniquet between an electrode and the current source is closed. Due to this possible embodiment according to the invention, premature activation of the TENS generator and of the stimulus current is prevented, so that a stimulus current can be applied only after the tourniquet has been attached in the intended position.

In another preferred embodiment of the device according to the invention, the tourniquet is characterized in that it has a base body having a strip-shaped elastic element. Reference is made to U.S. Pat. No. 5,738,398 as an example of this preferred embodiment, the content of said patent being herewith made part of the present description of a base body having a strip-shaped elastic element through this reference. In an especially preferred embodiment, the base body that has been described may be sheathed with a nonconductive elastic plastic, such that according to a preferred embodiment, the electrodes are connected to the base body. In another preferred embodiment, the electrodes may be attached directly to the tourniquet. In another possible embodiment, the base body may have a self-interlocking closure, by means of which the stasis capability of the device according to the invention can be increased. This may be accomplished, for example, by lateral pressure on the device according to the invention.

In addition, in the preferred embodiments of the invention, the device may include the fact that the tourniquet for venous collection of blood may have a variable width and may be made of an elastic deformable material, for example, a woven cotton latex strip, and may have a closure. In addition to latex, other elastomers may also be used here to form the tourniquet, for example, Neoprene®. In another embodiment, the elastomer used may be autoclavable in order to comply with medical hygiene requirements.

In addition, an embodiment according to the invention may comprise an intervention site situated in the tourniquet and optionally being of any desired design. In a preferred embodiment of the tourniquet, it may have a circular to oval shape and be of an extent sufficient to permit access to the intervention site for withdrawal of blood.

In a preferred embodiment, the tourniquet is designed with the electrodes so that the tourniquet may be varied in a flexible manner between linear rigid and circular flexible, as described in U.S. Pat. No. 5,738,398. In such an embodiment, an “intelligent” material, e.g., aluminum or some other flexible material is preferably used in particular. An “intelligent” material is also understood in particular to be a material that has a shape “memory” and can resume a shape “stored” previously with an appropriate action, for example, with an impact. In such an embodiment, the tourniquet, which is rigid in the starting position may be wrapped around the upper arm and then conforms to the upper arm through a change in shape. Then the tourniquet may be compressed to activate the stasis function. Such an embodiment allows more rapid use than the closures currently in use with the tourniquets known from the state of the art.

In a preferred embodiment, a material which is electrically conductive and has a larger area than the area used to form an electrode is used to form an electrode. It is preferable in particular for the entire tourniquet to be permeated by such a conductive material. The conductive material is covered with an insulating material and has an outlet the size of the area of the electrode only at the location where an electrode is to be formed.

The electrodes for the transcutaneous electrical stimulation may be self-adhesive in another embodiment of the device according to the invention such that an electrically conductive contact is formed by the resulting attachment of the electrodes for application of the stimulus current. Self-adhesive electrodes of this type are known in the state of the art. U.S. Pat. No. 4,458,696 describes the design of self-adhesive electrodes, which may be used for transcutaneous electrical stimulation. WO 94/27491, for example, describes the use of self-adhesive electrodes for application in the oral cavity; these electrodes may be used for transcutaneous electrical stimulation to suppress pain in dental or surgical procedures in the oral cavity.

In another embodiment of the device according to the invention, the electrodes are attached in the tourniquet in such a way that the tourniquet is attached on both sides of the intervention site according to the invention, preferably medially and laterally to the longitudinal axis of the respective body part. In another possible embodiment, the TENS device may be attached to the tourniquet. This attachment may be designed such that the tourniquet is provided with a holding device for the control unit and this is connected to the electrodes, which are then controllable, in an electrically conducting manner when introduced. The control unit here may also comprise the current source which may also be attached to the tourniquet, however.

To reduce or suppress acute and/or chronic pain, the invention proposes an electrode assembly, in particular for use in the aforementioned device. The two aforementioned electrodes in the tourniquet may be part of the electrode assembly. One surface of the electrodes is designed for contact with the skin and multiple anodes are provided. An electrode assembly having a simple design that can be controlled in a flexible manner may be created in this way. Due to the flexibility in controlling the electrode assembly, there is the possibility of a wide range of applications for pain reduction and/or pain suppression.

Multiple electrodes, i.e., a number greater than or equal to two, may preferably be provided. Furthermore, multiple cathodes may also be provided.

For the purpose of the present invention, a cathode and/or an anode is understood to be an electrode having a corresponding polarity. An electrode preferably does not change its sign when a current or voltage is applied. However, it is also possible to provide that an electrode is acted upon by current/voltage pulses having different polarities.

For the purpose of the present invention “contact with the skin” is to be understood to mean that the electrodes are preferably in contact with the skin and the skin is not perforated and/or is also not even partially penetrated by same. The electrode assembly may be worn on a human or animal body for contact with the skin. The electrode assembly may preferably be worn on the human or animal body without any significant restriction of the ability to move.

The electrode assemblies described here may preferably be used in the tourniquet, which is suitable for being attached to the body part and is capable of reducing the flow of fluid when applied to the body part.

The several anodes may preferably be designed as concentric anode rings around a central cathode. This arrangement permits a flexible application of power to the anodes. It is provided that at least one anode ring is controlled, but optionally two or more rings may also be controlled.

For a preferred embodiment, several anodes and several cathodes may be provided for a planar embodiment of the electrode assembly. The anodes and cathodes may be arranged in the form of an electrode assembly. In the case of the flat embodiment, the anodes and cathodes may be arranged linearly, which results in a simple geometry of the arrangement of the anodes and cathodes and at the same time provides a simple option for connecting the anodes and cathodes. For the purpose of the present invention, a linear arrangement is understood to be an arrangement in a row. Similarly designed electrodes are aligned with their midpoints as if on a chain. A linear arrangement also includes a wavy or zigzag-shaped arrangement. In another preferred embodiment, the anodes and cathodes may be arranged in alternation across the line, i.e., across the linear embodiment of the arrangement. In this way, a geometrically defined grid may be created with predefinable properties with regard to the emission of the electric field into the skin. It is possible to provide that with the alternating arrangement across the linear design, the cathode and anodes may be arranged more or less in a grid pattern directly opposite one another. It is also possible to provide that cathodes and anodes in a linear arrangement are offset in relation to one another, so that one cathode is opposite an interspace between two cathodes. An offset arrangement appears to reduce unintentional short circuits and can improve the pain reduction and/or pain suppression effect. A 40 mm×40 mm matrix having 17 rows and 17 columns may be provided, with the rows having an alternating sequence of anodes and cathodes. The matrix size and/or the size of the extent of the electrode assembly may be selected to be of any large or small size, depending on the field of application and/or the area of skin to be covered. For example, fabrics with an electrode assembly of 10 cm×10 cm may definitely be exceeded.

Mixed forms and/or combinations of flat electrode assemblies and electrode assemblies with a concentric electrode are also possible.

It is possible to provide that the electrode assembly comprises a conductive textile, which is embedded in a nonconductive textile and/or another material. It is possible to provide for the electrode assembly to comprise conductive fibers, which are incorporated in a nonconductive textile or some other material. This creates a device which is simple to handle and simple to manufacture. A carrier structure can be created by the nonconductive textile or other material. It is also possible to provide for a conductive structure comprising the electrode assembly to be printed on a textile or another material. It is also possible to provide for a structure or area which is designed to be nonconductive to be printed on a conductive material. The structure that is printed may be a flat structure but may also be a spherical structure. The spherical structure may have a geometry which varies in height. It is also possible to provide that the electrode assembly has at least one sphere which is knit or otherwise applied to a material or a textile. The examples given above permit simple integration of the electrode assembly into a bandage, for example. The electrode assembly may be integrated into the carrier or backing structure. Arrangements known from the medical field are preferably possible as the carrier structure such that there may be an adjustment between the electrode assembly and the carrier structure. It is also possible to provide that the electrode assembly is designed on a flexible circuit board and is embedded in a textile or some other material. In a preferred embodiment, the textile may have a dirt-repellant coating to ensure and/or improve contact with skin over a prolonged period of time.

It is possible to provide that the textile may perform a cold and/or heat stimulation of the skin of the animal body by means of the electrode assembly, in that the electrode assembly is acted upon by cold and/or heat. It is also possible to provide that cold and/or heat stimulation can be performed by cooling or heating elements in the textile regardless of the electrode assembly (cold pads and/or hot pads). Cold and/or hot stimulation is regarded as an improvement in the spectrum of effects.

The distance between an anode and a cathode next to this anode is preferably less than 3 cm. Through the choice of this distance, a targeted stimulation of certain nerve fibers is possible. In particular it may also be provided that the distance is less than 0.7 cm. Smaller distances of approx. 0.05 cm to 0.2 cm are also possible. Experiments by the applicant have shown that good results with regard to an improved pain reduction and/or pain suppression affecting in particular the nerve fibers of the superficial skin structure may be achieved for a distance of approx. 0.25 cm, for example.

The anodes and/or cathodes have a hemispherical surface for contact with the skin, so that in addition to a positive perception by the skin, the emission of the field into the skin is also improved. However, different anode shapes and/or cathode shapes are also possible. The anode and cathode shapes may be varied between acute, obtuse, rounded and angular.

The electrodes may preferably be designed as so-called ball grids. This is a circuit board on which a large number of small “balls” are placed on a surface. The circuit board is preferably flexible and therefore, together with the spherical shape of the electrodes, this permits an especially good contact with the skin. The balls may function first as the anode and cathode as well as being a means of transfer of vibration.

It is also possible to provide a unit for a change in temperature of the electrodes which are in contact with the human or animal surface. Then heat and/or cold pulses can be applied with the electrodes.

In a preferred embodiment the anodes may be arranged on a carrier structure so that good handleability can be achieved. The phrase “on a carrier structure” is preferably to be understood according to the present invention to mean that the electrode assembly is designed in or on the carrier structure. To improve the pain reduction and/or pain suppression that can be achieved, it is additionally possible to provide that the elevation of the anodes from the carrier structure is designed to be greater than the film of perspiration on the skin so that short circuits between the anodes and/or cathodes are prevented. The carrier structure may preferably be electrically insulated with respect to the anodes and/or cathodes to create the predefinable conditions for the electrode assembly. The carrier structure preferably has an electrically nonconductive material with which insulation of the anodes and/or cathodes can be achieved. The carrier structure may preferably comprise a material, which is absorbent and faces the skin while being insulated from the anodes and/or cathodes. In this way, defined conditions for introducing the pulses into the skin can be created by the fact that perspiration present on the skin can be absorbed and/or bound by the absorbent material. In a preferred embodiment, the carrier structure is designed in layers to achieve function associated with the individual layers.

To achieve a good adaptation to the skin contour, the carrier structure may be designed to be flexible in the direction of its surface normal. The electrode assembly “conforms” uniformly to the desired contour.

It may preferably also be provided that the anodes and/or cathodes have an adjustable and/or variable distance from one another. For example, it is possible to provide that the electrode assembly is designed as a displaceable grid electrode for adjusting the distances with an increase and/or a reduction in the carrier surface area.

In a preferred embodiment, the electrode assembly has a control unit with which the anodes together with the cathodes can be acted upon by electric pulses. The control unit may be set up at a distance from the body and the transfer of energy and/or control pulses may take place through a wireless standard (Bluetooth, Zigbee, etc.). Furthermore, the anodes may be acted upon by the control unit with electric pulses separately from one another with one or more cathodes, which increases the flexibility and parameterization of the electrode assembly and the pulse introduction possibilities achievable therewith.

The anodes can preferably be acted upon with a current of 1 to 15 mA in pulses that can be regulated by the control unit. The pulse period may be 100 μs to 500 μs. A value between 0.1 Hz and 10 Hz may be selected as the pulse repetition frequency. A pulse repetition frequency with a value between 50 Hz and 100 Hz may also be selected, which may denote a “burst stimulation,” i.e., an initial stimulation. The burst stimulation may preferably have a pulse repetition frequency with a value between 0.1 Hz and 10 Hz. The applicants have found a preferred value for the pulse repetition frequency to be 0.01 Hz to 5 Hz, at which the active spectrum has been found to be excellent. A battery is preferably provided as the power supply source for the control unit, requiring a maximum voltage in the two-digit volt range. A battery or a rechargeable battery which supplies a maximum voltage of approx. 9 volts is especially preferred.

It is possible in particular to provide that the current for application to the electrodes is regulated at a constant value, taking into account the pulse width. A certain energy may be applied in this way.

The control unit may be designed as an application-specific integrated circuit (ASIC) or a programmable microprocessor or controller. With the control unit it is possible to act on the electrodes in accordance with the setting. If the control unit is designed as a measuring and regulating unit, then it is possible to implement regulation for the parameters for applying current to the electrodes as a function of a measured or detected signal, for example, the skin resistance.

In a preferred embodiment, the electrode assembly can be arranged and/or placed on an animal or human body by using holding means. The holding means may be, for example, a patch, a material with an antistatic coating or a bandage. The holding means may be embodied as a holding device in which the electrode assembly is designed as a separable unit having an electrical connection for connecting the electrode assembly to a control unit on the holding device. It is also possible to provide that the electrode assembly is part of the holding means, but a control unit and/or a voltage supply are not part of the holding means. This makes it possible to create the option that the electrode assembly, which comes in contact with skin in accordance with its use, can be replaced easily after a certain service life. Furthermore, the possibility of cleaning the holding device without the electrode assembly can also be created. The control unit and the power supply may be accommodated in a housing where they are protected and where the housing can be manufactured by an injection molding technique, for example. The electrode assembly is more or less an arrangement having a limited or specific service life, which can be a function of the number of applications to the skin or of the number of possible cleaning cycles of the electrode assembly. For the purpose of the present invention, an electrical connection may be understood to be any releasable connection, which permits a rapid and secure connection between the electrode assembly and the control unit, preferably without a mechanical tool. The term “connection” should also include snap connections, plug connections, hook and loop connections, sliding connections and screw connections. The holding device and the electrode assembly may be designed so that the holding device with the electrode assembly does not cause any restriction on movement and/or with regard to accessibility to the body when worn on the human or animal body. The holding device for positioning the electrode assembly on the human or animal body may preferably have a thickness of less than 10 cm, in particular preferably a thickness of less than 5 cm and most especially preferably a thickness of less than 3 cm.

It may preferably be provided that at least one vibration motor, which can be connected to the control unit by a plug connection, is attached to the unit. The vibration motor may be, for example, a PicoVibe™ vibration motor. A vibration motor having a voltage supply with a nominal voltage of 1.5 V is especially preferred. A typical vibration amplitude of 1.2 G is especially preferred. It is possible to provide for the typical power consumption to be 150 mW with an operating current of 100 mA.

According to the invention, a use in which an electrode assembly is used to stimulate nerve fibers in a human or animal to suppress or reduce a chronic or acute pain is also proposed, such that the electrode assembly can be brought into contact with the skin of the person or animal and several anodes are provided with the electrode assembly.

Tests by the applicant have shown that the aforementioned electrode assembly can be triggered in particular for achieving or triggering an LTD on synapses of nerve cells to reduce acute and chronic pain states.

It has been found that for achieving or triggering an LTD, the TENS may not be sufficient. Therefore a device and a method with which an LTD can be triggered on synapses of nerve cells to reduce acute and chronic pain states is also being proposed.

Thus a device for use on the surface of a human or animal body with an electrode assembly and a method for reducing chronic pain, acute pain, itching, incision pain and/or wound pain on a human or animal body with an electrode assembly is also proposed. One embodiment of the invention is a device for inducing a long-term reduction in signal transmission to the synapses of nerve cells (long-term depression) in the human or animal body with the help of electromechanical pain suppression (EMPS). Acute or chronic pain states as well as itching can be reduced in this way. The mechanism may also be used as electromechanical analgesia topically in minor procedures or in treating wound pain.

The nociceptive system has two types of pain fibers: Aδ and C nerve fibers. The Aδ fibers are relatively thick (approx. 3-5 μm) and are the fast-conducting (5-50 m/s) type of nerve fibers because of their sheathing, the so-called myelin sheath. However, the C fibers are thin (approx. 1 μm) and are not myelinized. They have stimulus conduction speeds of less than 1 m/s. Aδ fibers end in strata I and V of the posterior horn of the spinal cord, where they form synapses with projection neurons. However, most C fibers are connected to interneurons in stratum II. The axons of the touch and temperature sensors end in strata III and IV of the posterior horn.

These two populations of pain fibers create a time lag in pain perception: the first pain, which is perceived in fractions of a second, is conducted by the Aδ fibers and is often stinging or burning. A few seconds later, the pain mediated by C fibers arrives; this pain is described more as piercing or nagging.

It is known that transcutaneous electrical nerve stimulation (TENS) uses electro-medical stimulus current therapy with square-wave pulses of a low frequency, i.e., 2 to 4 Hz, or a high frequency, i.e., 80 to 100 Hz. TENS is used mainly for treatment of pain and for muscle stimulation. Electric pulses are transmitted via electrodes to the surface of a human or animal body. The electrodes are usually placed near the painful locations.

If there is a long-term stimulation of the two types of pain fibers described above, this leads to so-called long-term potentiation (LTP) at the level of the spinal cord. Experiments have shown that this long-term potentiation disappears as a result of frequency-specific stimulation of the afferent nerve fibers. Long-term inhibition or long-term depression (LTD) of the synaptic transmission can be induced by low frequency electric stimulation. To reduce chronic pain in particular, the neurobiological pain mechanism of sensitization of central pain neurons in the posterior horn of the spinal cord (central sensitization) is to be regulated by an electrical stimulus as “counter irritation” (LTD). Central sensitization of pain processing involves a pathological mechanism that occurs with all possible pain conditions. On the other hand, LTD involves a spinal learning mechanism (localized in the spinal cord) which leads to a down-regulation of pain sensitivity, so that a pain sensitivity that has previously been increased, e.g., in conjunction with a gonarthrosis, is reduced again. This achieves an improved mobility, endurance under everyday conditions and thus an increased quality of life.

It has been found that a preferred embodiment for pain reduction and pain suppression according to the present invention involves an electrode assembly, which is made to vibrate, thereby enabling the transmission of periodic vibrations of a medium to high frequency and a low amplitude to the skin surface, so that the electrode mechanically stresses the skin surface to achieve an optimal vibration capability. It is possible in this way to stimulate various nerve fibers (for example, Aβ fibers), which in turn can interrupt the conduction of pain by the Aδ and C fibers (modified gate control hypothesis).

In a preferred embodiment, the electrode assembly is thus vibrated, so that primarily myelinated mechanoafferences (Aβ fibers) are stimulated.

According to the invention, “contact with the surface” is understood to mean that the electrode assembly rests essentially on the skin of the person or animal. However, in the case of a wound in particular it is possible to provide that the electrode assembly for wound care comes in contact with the wound, i.e., the injured skin.

Changes occur in the spinal nociceptive information inputs due to specifically adapted stimulation patterns at several levels of the pain processing system. The substantia gelatinosa in the posterior horn of the spinal cord takes over the function of a “gate” for incoming nociceptive information. Aβ fibers can have inhibiting effects on the incoming pulses of the Aδ and C fibers in the posterior horn. This results in modulation at the substantia gelatinosa, which in turn leads to activation of the inhibitory interneurons. With a normal pain stimulus, activation of the excitatory interneurons on the substantia gelatinosa is triggered. Finally the typical pain occurs as a result of various switching mechanisms.

The effect of the LTD described above can be triggered by various pulse patterns between 0.1 and 20 Hz. This is differentiated fundamentally from previous/related approaches based on TENS technology (transcutaneous electrical nerve stimulation) and provides greater depth to the latest scientific findings in this field. The electrode shape that is described in greater detail below achieves merely superficial stimulation of the cutis and subcutis with the Aδ and C nerve fibers running through these layers. In addition to the special electrodes, this requires an integrated measurement and control technology which ensures a parameter adjustment for each specific situation and each patient. The electric current required for stimulation of a myelinized nerve by skin electrodes preferably have a monophasic square wave pulse with a period of approximately 0.05 to 3 ms which can be varied.

Apart from the amplitude, the pulse width also plays a crucial role in the fiber spectrum that is excited. The shorter the pulse, the higher must be amplitude and vice versa. To stimulate the large caliber fibers required for pain relief, an adequate pulse width, which is usually fixed and is usually approximately 0.2 to 0.3 ms is used to create the prerequisite for being able to reduce the amplitude. A square-wave pulse is preferably used as the pulse shape.

It is also possible to provide that the pulse shape has a sinusoidal characteristic.

The electrode assembly is preferably an electrode array. According to the invention, an electrode assembly is understood to be an arrangement of electrodes embodied over an area. Multiple electrodes are provided in an area between approximately one and several square millimeters. Thus, for example, areas between 1 cm×1 cm and 10 cm×10 cm are possible. A square area is preferred, but rectangular areas are also covered by the present invention. It is preferable in particular for the area arrangement of electrodes to be designed to be flexible with regard to the surface of the electrodes, which comes in contact with the surface of the human or animal body, so that the electrode assembly may be applied to the surface of a human or animal body without any loss of contact. The arrangement of electrodes in the electrode assembly is such that anodes and cathodes are arranged next to one another alternating in a line in the electrode assembly. In the next line of the electrode assembly, similar electrodes, i.e., anodes and cathodes are arranged side by side. The distance between a cathode and a neighboring anode is preferably in the range of 1.5 mm to 3.5 mm, the distance between the immediate neighboring anode and cathode preferably being 2.5 mm in particular. The contact surfaces may be selected to be as short as possible so that a pulse can be applied beneath the secretion film of the skin. This achieves effective stimulation of the nerve fibers running in the cutis and subcutis. Muscular stimulation is almost ruled out because of the brief current flows between the anodes and the neighboring cathodes.

The electrodes may preferably be designed as so-called ball grids. This is a circuit board with a large number of small “balls” placed on a surface. The circuit board is preferably flexible and therefore an especially good skin contact is possible together with the spherical shape of the electrode. The balls function as the anode and cathode and also function as the means of transmission of the vibration.

Furthermore, it is possible to provide for a unit for a change in temperature of the ball grid and/or the electrode surfaces in contact with the human or animal surface to be present. Then heat and/or cold pulses can be applied with the electrodes and/or the ball grids. Furthermore, the ball grids may be designed as measurement heads of a measurement and control unit with which a skin resistance can be detected. Thus an optimal effect can be achieved by automatically varying the pulse intensity.

It is also possible to provide that the electrode assembly has at least one concentric electrode which comprises a central cathode, preferably with a narrow design and a larger anode preferably in a ring shape surrounding the cathode. It is also possible to provide that the cathode is split into a large number of small cathodes arranged centrally. It is also important here for the distance between the two electrodes not to be greater than 3.5 mm, and in particular it is preferably smaller than 2.5 mm. It is also possible to provide that the anode is designed centrally and the cathode surrounds the anode. The electrode may be manufactured to be flexible or solid. The anode and the cathode are preferably designed in a ring shape, so that the cathode has a diameter in the range of a few millimeters, the diameter preferably being 1 mm. The anode preferably has an inside diameter of approximately 8 mm and an outside diameter of approximately 24 mm.

However, it is also possible to provide that only one electrode is provided and that the current is diverted to a reference electrode.

Stimulation with a frequency of 0.1 to 20 Hz is possible by means of this electrode assembly. The effect of LTD is therefore triggered in an optimized manner.

In a preferred embodiment the device has a measuring and control unit which permits detection of the current resistance of the skin and/or the impedance in the stimulation pauses. With a feedback loop, the pulse emitted by the electrode assembly can be adapted based on the current skin resistance detected and/or the impedance. The measurement and control unit thus detects the prevailing skin resistance and/or the impedance and uses the value thus detected as a controlled variable for controlling the electrode assembly. The secretion status of the skin can be ascertained in this way and used as a controlled variable.

The conductors of the electrode assembly or assemblies are preferably designed to be flexible or they have gel electrodes and/or textile electrodes. It is thus possible to provide that a gel matrix includes the electrode assembly which is either elastic itself or includes individual parts or components in an elastic connection in which all the conductors are made partially or completely of a conductive medium (liquid, electrolyte, metallic conductor). Thus the electrode is constructed like a gel pad or a blister pack. The compressible filling allows an especially good contact with the skin. Due to the vibration device for vibration of the electrode assembly, vibrations can be transmitted via the liquid medium especially well directly to the skin surface. In the case when the electrode assembly has textile electrodes, a conductive textile is used in which all the printed conductors are either printed or woven as a conductive material. This implementation allows especially good wearing comfort as well as good contact with the skin surface. Furthermore, especially good insulation of the body part is achieved through the textile for which neoprene, for example, may be used. Skin secretions, i.e., perspiration can be absorbed well through the textile, so this reduces the risk of short circuits between the anode and cathode. However, it is also possible to provide that the electrode assembly has solid conductors. The contact elements, i.e., the outer surface of the electrode assembly facing the surface of the human or animal body may be supported elastically with respect to the solid conductor. This may be embodied, for example, by a spring mechanism by means of which the contacts, the so-called pins, sitting on a spring or a spring arrangement can be pressed into a matrix. The electrode assembly can also be placed on curved surfaces of the human or animal body in this way.

The electrode assembly is preferably provided with a coating of a pharmacological substance class so that in addition to the stimulation, an additional effect can be achieved, for example, for wound healing. The coating is such that the electrodes of the electrode assembly that come in contact with the human or animal body are coated on one end. The substance classes may include, for example, local anesthetics, substances containing cortisone, etc.

In a preferred embodiment, the power supply to the device may be provided by energy harvesting. To do so, a device for generating energy which is connected to the electrode assembly may be provided. The primary goal in energy harvesting with respect to the present patent application is to effectively operate the pulse generating unit with energy for supplying electric voltage to the electrode assembly and operating the vibration unit for vibration of the electrode assembly. The energy required for operation of the device may be obtained at least partially by utilizing the difference in temperature between the skin and the surroundings, for example. It is also possible, for example, to utilize the kinetic energy generated by movement by the user, for example. To utilize the kinetic energy, it is necessary to implement the device in the form of bandages, orthotics or supporting units. The kinetic energy may be mechanical—like the winding mechanism with automatic watches. The charging may take place wirelessly by induction. It is possible to provide for the device to have a battery or a rechargeable battery which is charged by energy harvesting. The battery or the rechargeable battery may be connected to the pulse generator unit and the vibration unit for the power supply.

The device—and in particular the partial area of the device having the pulse generating unit—is preferably protected from exposure to perspiration and washing operation because the device may be used in bandages that must be washed and the device is exposed to moisture, dirt, chemicals and/or the action of forces because of the area of application during use. It may be preferable for the circuit for providing electricity to the electrode assembly to be permanently welded. In a preferred embodiment a cushion—for example, in the form of a gel pad—is provided, buffering and/or reducing the action of force on the circuit.

The present invention also creates a method for reducing product pain, acute pain such as itching, incision pain and/or wound pain, for example, in a human or animal body with an electrode assembly. Surface stimulation of the cutis and subcutis is performed by the electrode assembly. The electrode assembly is vibrated in performing the surface stimulation.

In this method the secretion of perspiration is determined in order to regulate the pulses emitted by the electrode assembly via the pulse generating unit by means of a measurement and control unit.

Energy is preferably produced for application to the electrode assembly. The energy is produced in such a way that a conversion unit is provided in proximity to the electrode assembly, converting mechanical or thermal energy into electric energy. The electric energy may be stored temporarily in a battery or a rechargeable battery.

In a preferred embodiment stimulation is triggered for a period of time of more than 1 minute at a frequency of 0.1 to 20 Hz.

### BRIEF DESCRIPTION OF THE FIGURES

The invention is explained in greater detail below on the basis of drawings which show only possible embodiments of the invention, which show:

FIG. 1: a schematic diagram of attaching the device according to the invention to an arm and a cross section through the upper arm in the plane of attachment of the tourniquet,

FIG. 2: a possible embodiment of the tourniquet according to the invention together with a possible arrangement of the stimulus current electrodes,

FIG. 3: a possible embodiment of the invention comprising an intervention site in the tourniquet.

FIG. 4: a possible arrangement of stimulus current electrodes and control unit connected to the electrodes attached to the tourniquet.

FIG. 5A: a tourniquet having sections connectable by a closure such that when closed, an electric line provided in the tourniquet between an electrode and the current source can be closed,

FIG. 5B: the tourniquet according to FIG. 5A after attaching it to the upper arm and closing the closure,

FIG. 6: another embodiment of the device according to the invention in a schematic diagram,

FIG. 7: a schematic diagram of another embodiment of a tourniquet before applying it to a body part,

FIG. 8: a schematic diagram of the device according to the invention according to FIG. 7 after applying it the upper arm,

FIG. 9: a schematic diagram of an electrode assembly according to the invention which may preferably be used in a device according to one of FIGS. 1 through 8,

FIG. 10: a schematic diagram of a device according to the invention,

FIG. 11a: a schematic to view of an electrode assembly of the device according to FIG. 10,

FIG. 11b: the electrode assembly from FIG. 11a in a schematic perspective view,

FIG. 12: an alternative exemplary embodiment of the electrode assembly of the device according to FIG. 10,

FIG. 13: mechanical pain threshold at 1 Hz,

FIG. 14: mechanical pain threshold at 3 Hz,

FIG. 15: mechanical pain threshold at 4 Hz,

FIG. 16: pressure pain threshold (PPT),

FIG. 17: a spiny electrode,

FIG. 18: a function pattern,

FIG. 19: test area,

FIG. 20: pain threshold with BASELINE (days 1, 2), SHAM (days 3, 4) and EMPS (days 5, 6),

FIG. 21: PPT results of test area 1 (medially),

FIG. 22: PPT results of test area 2 (patella) and

FIG. 23: PPT results of test area 3 (laterally).

### DETAILED DESCRIPTION

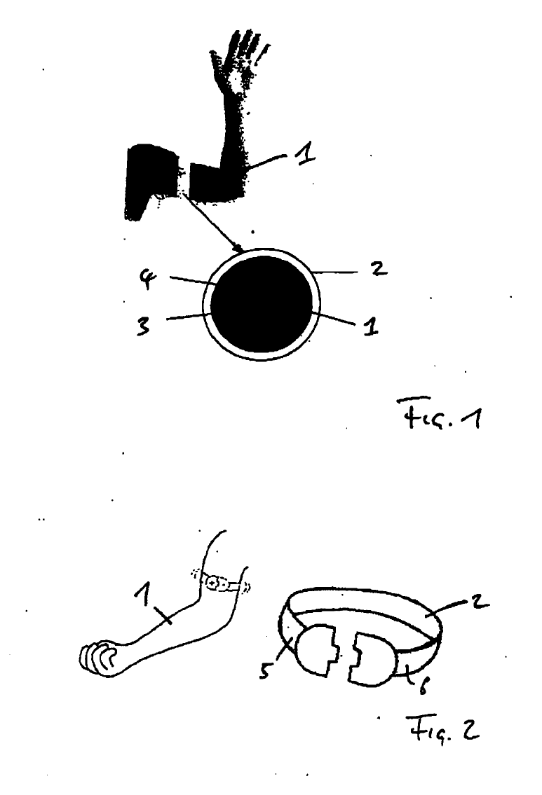

FIG. 1 shows a schematic diagram of the attachment of the device according to the invention to an arm 1. In the cross-sectional view through the upper arm 1 in the plane of attachment of the tourniquet, also shown in FIG. 1, the tourniquet 2 in the form of a tourniquet is shown in its attachment to the upper arm 1. This shows that the upper arm 1 has a bone 3 and a vessel 4, i.e., a vein in which the blood has been brought to stasis.

The embodiment of the tourniquet 2 belong to the invention as illustrated in FIG. 2 shows that the tourniquet 2 has sections 5, 6 that can be connected by a closure. By connecting the sections 5, 6, an electric line which is provided in the tourniquet 2 (not shown in detail here) is closed as a circuit between an electrode (not shown in detail) and a current source (also not shown in detail). In the diagram of upper arm 1 which is also shown in FIG. 2, illustrating how the device according to the invention is attached to the upper arm 1 in this embodiment. The plus and minus signs shown in the sections of the closure to be joined indicate that an electric line can be closed here as a circuit by connecting these sections.

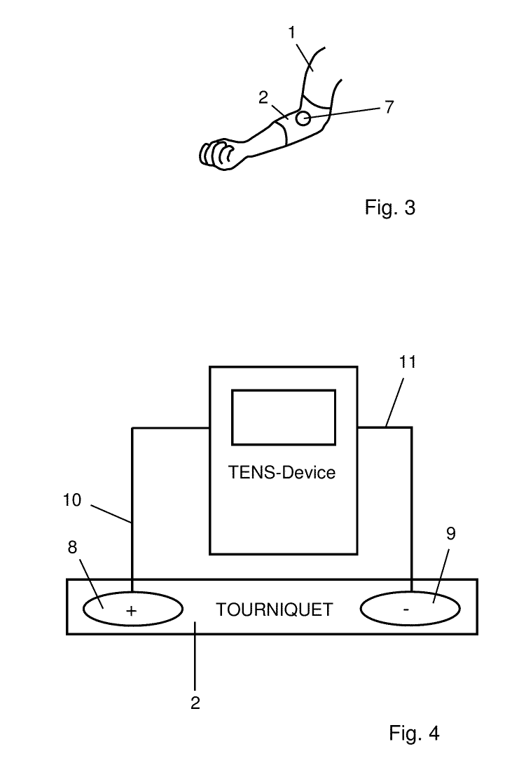

FIG. 3 shows another embodiment of the device according to the invention where the tourniquet 2, as a continuous cuff, is provided with a recess for the puncture site 7 (intervention site). The electrosimulation may be activated by means of a button. This type of bandage support may also be used for minor surgical procedures and for pain reduction in the joint.

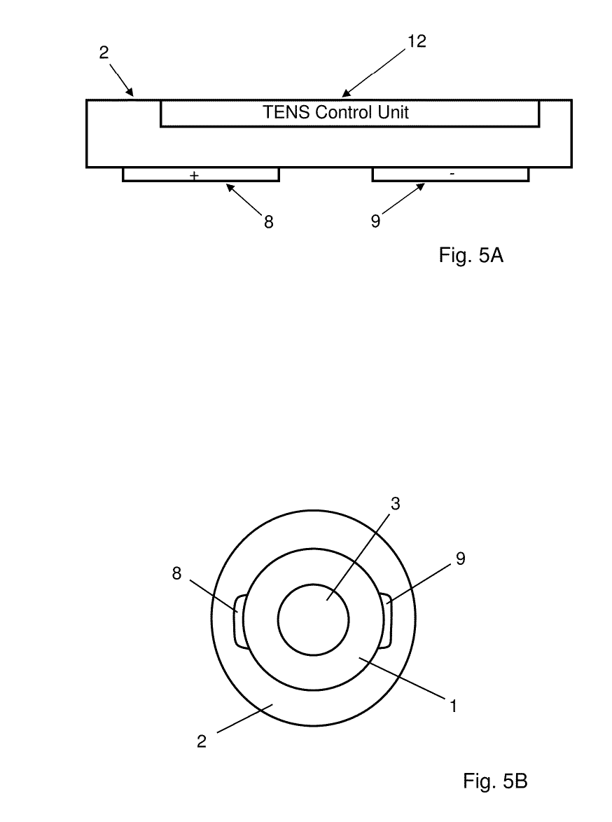

FIG. 4 shows one possible design of the tourniquet 2 to be used. This tourniquet has two electrodes 8, 9. The electrodes 8, 9 are secured in small pockets (not shown in detail) in the tourniquet 2 which is made of a flexible material. The electrodes used have a minimum size of 40 times 40 mm and are coated with a conductive medium (self-adhesive). Furthermore, the device according to the invention in the embodiment shown in the figure have a control unit (TENS device) which is connected to the electrodes 8, 9 by electric conductors 10, 11. The control unit is designed to be as small as possible and may preferably be secured in the tourniquet or on the tourniquet 2 in particular, for example, being secured in one of the sections of a closure. Such an arrangement is illustrated in FIG. 5A where the TENS control unit 12 is arranged in one layer of the tourniquet 2. The electrodes 8, 9 are designed in a second layer of the tourniquet 2. When such a tourniquet 2 is placed around the upper arm, as illustrated in FIG. 5B, the electrodes come in contact with the upper arm.

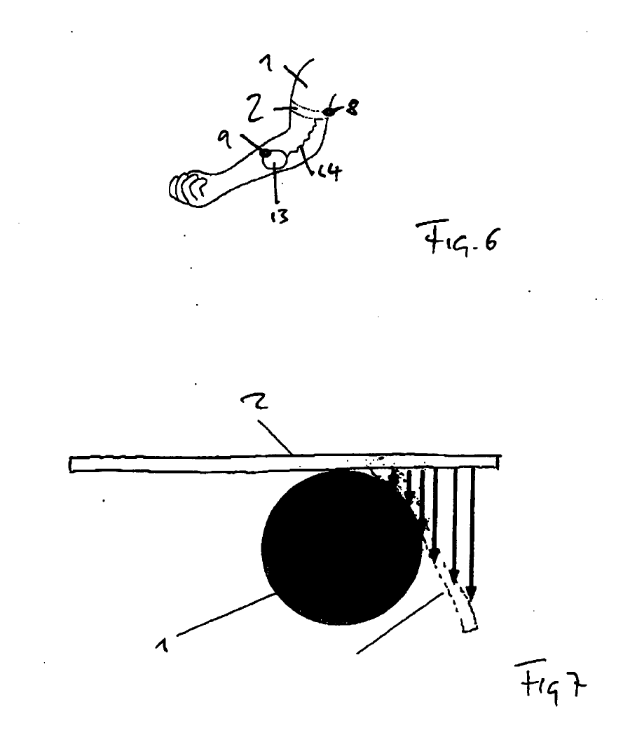

An arrangement in which the electrode 8 is arranged on the tourniquet 2 but the electrode 9 is designed to be separate from the tourniquet 2, namely on a bandage or patch 13, was selected in FIG. 6. The electrode 9 is connected by a line 14 to a control unit which is formed on the tourniquet 2 but is not shown in detail here.

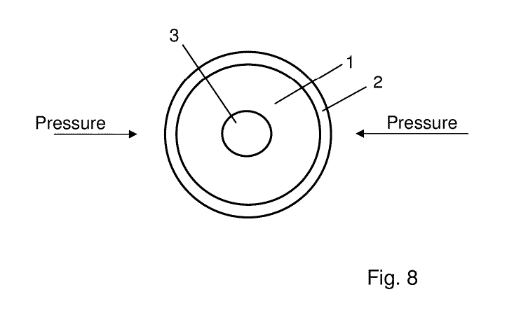

FIG. 7 shows another embodiment of the device according to the invention, where the tourniquet 2 is designed as a strip-shaped elastic element. As indicated in the dotted line variant in FIG. 7, the strip-shaped elastic element yields after being struck and conforms to the shape of the upper arm. Such devices are designed according to the type of device presented in U.S. Pat. No. 5,738,398. A tourniquet can be applied to upper arms of various sizes in an especially favorable manner in this way. FIG. 8 shows the tourniquet from FIG. 7 after being attached to the upper arm. By pressing laterally on the tourniquet 2, the tourniquet can be further compressed so that the stasis capacity can be varied.

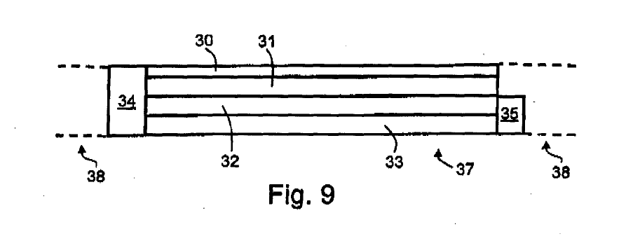

FIG. 9 shows schematically an electrode assembly according to the invention, which may preferably be used in a device according to any one of FIGS. 1 through 8. This shows an electrode assembly with electrodes 30 having a plurality of anodes and at least one cathode which can come into contact with the skin of a human or an animal. The electrodes 30 are arranged in or on a carrier structure 31 which is preferably designed to be allergen-free. The carrier structure 31 may also have nubs of silicone and/or silicone-coated nubs. On the side of the carrier structure 31 opposite the electrodes 30, the carrier structure 31 is mechanically connected to a mechanical applicator which is designed in the form of a vibration motor 32. Vibration motor 32 is designed as a vibration generator to apply vibrations to the skin via the electrodes 30 and/or the nubs on carrier structure 31. The vibration can be transferred to a large area due to the carrier structure 31 and additional elements mechanically connected to it. A flexible outer layer 33 is provided on the rear side of the vibration motor 32. The electrodes 30, the carrier structure 31, the vibration motor 32 and the outer layer 33 together form a unit 37 which can be handled as a whole.

For operating the electrode assembly and/or electrodes 30 and the vibration motor 32, a voltage source, i.e., a power source 34 is provided and can be electrically connected to the electrodes 30 and the vibration motor 32 in the form of a plug connection. The electrode assembly and/or the electrodes 30 and/or the vibration motor 32 may be controlled via a control unit 35 which is designed as a measurement and control electronic unit. The control unit 35 can apply pulses to the electrodes 30 of the electrode assembly.

The unit 37 may be inserted into a holding device 38 which is indicated schematically with the dotted lines—and is designed as the tourniquet in the embodiment shown in FIGS. 1 through 8. The control unit 35 and the power supply 34 may be part of the holding device 38. The power supply 34 is preferably detachably connectable to the holding device 38 to allow a replacement of the power supply 34, for example, in the event of a defect. The holding device 38 may have a(n) (active) bandage or (active) bandage-type design.

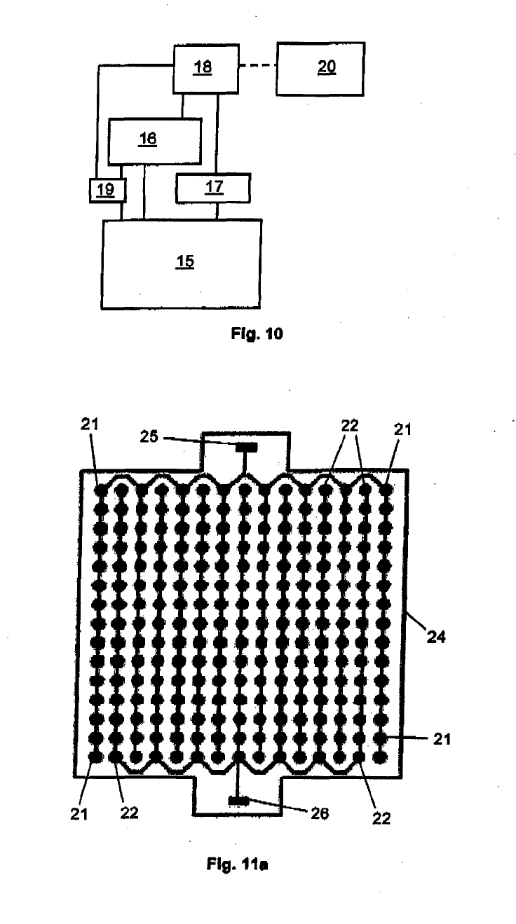

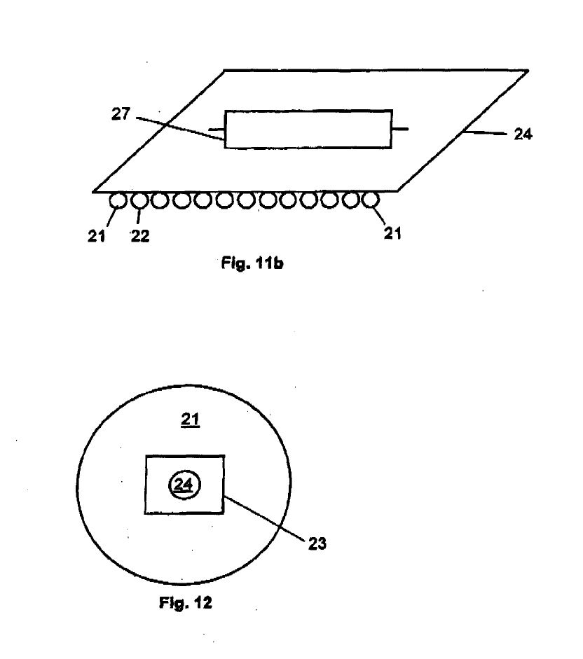

FIG. 10 shows schematically and device according to the invention. This device is provided for use on the surface of a human body and therefore has an electrode assembly 15 which may come in contact with the surface of the human body. The surface of the electrode assembly 15 comprises the ends of electrodes. A pulse generating unit 16 is operatively connected to the electrode assembly. The electrode assembly 15 may receive a voltage and/or electric current from the pulse generating unit 16. Furthermore, the device has a vibration unit 17 for vibration of the electrode assembly 15. The vibration unit 17 may be physically embodied in the electrode assembly 15 and/or may be physically connected to the latter. A device 18 for supplying energy is provided for the power supply to the pulse generating unit 16 and the vibration unit 17 and is therefore connected to the pulse generating unit 16 and the vibration unit 17. The device 18 for supplying power may be embodied as a battery or a rechargeable battery, supplying electric voltage and/or current to the pulse generating unit 16 and the vibration unit 17.

According to FIG. 10, this device also has a measurement and control unit 19 with which the prevailing skin resistance and/or impedance can be determined on the electrode assembly 15, for example. The value detected by the measurement and control unit 19 is used as a controlled variable such that the measurement and control unit 19 influences the pulse generating unit 16 connected to it in such a way that the duration of the pulses and the intervals between the pulses is/are regulated and/or altered as a function of the size of the measurement value detected. The measurement and control unit is connected to the device 18 for supplying energy for the purpose of an electric current and/or a voltage supply.

Furthermore, FIG. 10 shows a device 20 for converting thermal and mechanical energy into electrical energy as part of the device in one exemplary embodiment with which the device 18 can be supplied with electric power for providing energy. The dotted line between the device 18 for supplying energy and a conversion device 20 indicates that the connection may also be wireless, i.e., in this case inductive.

FIG. 11a shows schematically a top view of the electrode assembly 15 of the device according to FIG. 10 in a first exemplary embodiment. This view is such that it is a view of the surface of the electrode assembly 15 which comes in contact with the surface of the human or animal body. The electrode assembly 15 comprises spherical electrode ends on a circuit board 24 designed as anodes 21 and cathodes 22. The circuit board 24 is flexible. The arrangement of the cathodes 22 and anodes 21 is flat in the form of an electrode assembly, in which anodes 21 and cathodes 22 alternate in a line in direct proximity to one another. In one line which extends at 90° to the first line, similar electrodes are arranged side-by-side and electrically connected to one another. An anode connection 25 and a cathode connection 26 are provided on a continuation of the circuit board 24 outside of the area of the electrode assembly for connecting the anodes and/or cathodes. There is an odd number of both anode and cathode rows, i.e., the lines in which similar electrodes are arranged side by side and electrically connected to one another, to ensure a uniform current distribution. The number of anode rows and cathode rows is thus 2n+1, where n is a natural number. The number of anode rows is n and the number of cathode rows is n+1 or vice versa. If the anode and cathode rows are not an odd number, then the last electrode row would have to receive a voltage twice as high because otherwise the current between the last electrode row and the next-to-last electrode row would be only half as large.

FIG. 11b shows schematically the electrode assembly 15 according to FIG. 11a in a perspective view. The ends of the anodes 21 and the cathodes 22 of the electrode assembly are in the form of beads. A vibration motor 27 is shown, contacting the circuit board 24 with the anodes 21 and the cathodes 22. Contacting of the vibration motor 27 with the circuit board 24 may be such that the longitudinal ends of the vibration motor 27 are each connected to the circuit board 24.

FIG. 12 shows an alternative embodiment of the electrode assembly 15 of the device according to FIG. 10. A narrow central ring-shaped cathode 22 is formed inside an anode 21, which is also designed in a ring shape. The end surfaces of the anode 21 and of the cathode 22 may come into contact with the surface of the human body. A receptacle 23 for the cathode 22 is arranged between the anode 21 and the cathode 22, the cathode being mechanically connected to the vibration device 17 for vibration of the electrode assembly 15. The anode 21 which is designed in ring shape has an inside diameter of 8 mm and an outside diameter of 24 mm. The narrow central cathode 22 has a diameter of 1 mm.

The invention is described above with reference to a few embodiments. Those skilled in the art will recognize that overlaps and combinations of the embodiments may be intentional and desired. For example, the embodiment described above and illustrated in FIGS. 9 through 12 may be combined. Parameter settings for application to the electrodes—for example, pulse period, frequency, amplitude, etc. —which are described with respect to one embodiment may also be used for embodiments for which this is not specified explicitly. Furthermore, it is possible to place the electrode assembly according to the invention on the skin by using a variety of holding means, i.e., preferably by means of a (wound) dressing, a material with an antistatic coating (where the material with the antistatic coating may also be designed as a dirt-repellant coating), a bandage or a strip-shaped textile, etc. The electrode assembly may also be part of the holding device. The embodiments in FIGS. 9 through 12 may preferably be used in an embodiment illustrated in FIGS. 1 through 8.

An electrode assembly according to the invention may preferably have a flat arrangement of electrodes, an electrode arranged concentrically around a central electrode or a combination thereof. All the electrode assemblies may preferably have a weight of less than 500 g, more preferably less than 100 g, in particular preferably less than 20 g. The electrode assembly may be designed for contact with the skin. It may also be provided that the electrode assembly has electrodes which penetrate through or perforate the skin. The flat arrangement of electrodes, the arrangement of electrodes concentrically around a central electrode or a combination thereof may be designed so that the distance between an anode and a neighboring cathode to the anode is less than 3 cm, but the values given above are also possible. Electrodes of all the aforementioned electrode assemblies may also be formed on a circuit board which is designed to be flexible. The electrode of the flat electrode assembly, the concentric electrode assembly and mixed forms thereof may have a hemispherical surface for contact with the skin. All the aforementioned electrode assemblies may be arranged on a carrier structure. The electrodes formed on a circuit board which may be designed as a ball grid may be introduced into a carrier structure and/or arranged thereon. The elevation of the electrodes from the carrier structure may be designed to be greater than the thickness of the film of perspiration on the skin. The carrier structure may comprise an electrically nonconducting material by means of which the electrodes are insulated with respect to the carrier structure. The carrier structure of the flat electrode assembly, concentric electrode assembly or combination of the two electrode assemblies may comprise a material which is absorbent and faces the skin. For all electrode assemblies the carrier structure may comprise materials arranged in layers. The carrier structure may be designed to be flexible for all electrode assemblies. The spacing of the electrodes of all electrode assemblies may be adjustable. The electrode assemblies may receive electric pulses from a control unit. The control unit may be designed on the carrier structure or at a distance from the body. The electrodes of all the electrode assemblies may receive pulses so that a regulable current of 1 to 15 mA is preferably used. A period of 100 μs to 500 μs may be used as the pulse period for all electrode assemblies. A pulse repetition frequency of 0.1 Hz to 10 Hz may be used for all electrode assemblies. A different pulse frequency is possible as the initial stimulation (burst stimulation). A battery which is available for a voltage of 9 V may be used as the voltage supply source for the electrode assemblies. A programmable microprocessor may be provided as the control unit for the electrode assemblies. A holding means may be provided for the electrode assembly, i.e., the flat electrode assembly, the concentric electrode assembly and mixed forms thereof. The holding means may be designed as a textile, a bandage, a material with an antistatic coating, etc. The control unit for the electrode assemblies may be designed permanently or detachably in the holding means. The electrode assembly may be fixedly or detachably connected to the holding means and/or integrated into the holding means. All the electrode assemblies may be used in particular to reduce the wound pain.

All the electrode assemblies may be set in vibration by means of a vibration motor. To do so, the electrode assembly may be mechanically connected to a vibration motor. The applicants have found that a vibration unit allows a pleasant, low frequency stimulation, but it also stimulates additional nerve fibers (Aβ fibers) which potentiate the mechanism of action already described above. Low frequency stimulation produces a weak activation of NMDA synapses and leads to a slight increase in the intracellular calcium concentration. This produces a long-term depression (LTD) by means of a corresponding cell mechanism. The so-called NMDA receptor-dependent LTD (NMDAR-LTD) and metabotropic glutamate receptor-dependent LTD (mGluR-LTD) are the mechanisms known in the past which can be addressed here. In NMDAR-LTD, the so-called NMDA receptor is activated by simultaneous transmitter secretion at the synapses and electrical stimulation, so that calcium can flow into the cell. As an intracellular signal molecule, calcium also activates a series of other enzymes in LTD, said enzymes regulating signal transmission to the synapses. Metabotropic glutamate receptors are activated in mGluR-LTD, so that as so-called G proteins, they trigger the secretion of calcium from intracellular reserves by means of additional signal molecules. Both types of LTD may occur on the same synapses but they make use of separate mechanisms. The applicants have been able to ascertain that pain memory can be influenced by long-term stimulation with the help of a bandage, for example. The mechanisms mentioned above can lead to a reduction in or normalization of pain states.

The control unit can supply electric currents and/or voltage to the vibration motor. The control unit may be embodied as a measurement and control unit for all electrode assemblies so that the perspiration secretion can preferably be determined. The electrodes of the electrode assembly may comprise gel electrodes or textile electrodes. At least one of the electrodes of the electrode assemblies may be provided with a coating of a pharmacological substance class. Energy for application to the electrode assemblies can be obtained for each of the aforementioned electrode assemblies.

Three investigational studies which demonstrate the efficacy of the embodiments according to the invention are presented below in the form of examples.

### First Example

Investigation of the effect of pain suppression according to the invention on various pain modalities by means of quantitative sensory testing (QST)

This study shows that use of the electrodes according to the invention leads to an increase in the pain threshold when experiencing pain. This may cause a long-term depression (LED) of the synaptic transmission due to low-frequency electrical stimulation.

### Method

On the basis of a protocol for quantitative sensory testing (QST, Rolke, R., Magerl, W., Campbell, K. A. et al., 2006, Quantitative sensory testing: a comprehensive protocol of clinical trials, European Journal of Pain, Vol. 10(1), pages 77-88; Rolke, R., Baron, R., Maier, C., et al., 2006, Quantitative sensory testing in the German Neuropathic Pain Research Network (DFNS): Standardized protocol and reference values, PAIN, Vol. 123(3), pages 231-243; Backonja, M. M., Walk, D., Edwards, R. R., Sehgal, N., Moeller-Bertram, T., Wasan, A., Irving, G., Argoff, C., Wallace, M., 2009, Quantitative sensory testing in measurement of neuropathic pain phenomena and other sensory abnormalities, Clinical Pain, Vol. 25, pages 641-647; Rolke, R., 2009, Diagnostic “workup” of neuropathic pain in clinical practice: Quantitative sensory testing as a complementary method to conventional electrophysiology, Klinische Neurophysiologie [Clinical Neurophysiology], Vol. 40, pages 177-182), the influence of two different electrode assemblies according to the invention is tested. The goal of this testing is a standardized investigation of two symmetrical body areas. The stimulated skin area is always investigated before the control side on the volunteers. Ninety minutes is usually the length of time required to perform the entire protocol for two areas.

### Sequence and Design:

The local stimulation is performed using the electrode assembly according to the invention (see below) on the forearm and connecting them to a stimulus current generator. The test areas on the right or left forearms with the electrode assembly according to the invention: a concentric electrode with a frequency of 1 Hz/5-minute stimulation duration or a spiny electrode with frequency of 4 Hz/15-minute stimulation duration; intensity up to 2.5 mA. The stimulation is performed on a randomized basis, either as the verum or as a placebo with an ineffective stimulation pattern for 5 minutes (concentric electrode) and 15 minutes (spiny electrode).

### Design of the Electrodes

1. Concentric electrode: Large external ring anode with an internal diameter of 8 mm and an outside diameter of 24 mm. Narrow central cathode with a diameter of 1 mm.

2. Spiny electrode (matrix array): This spiny electrode is a multielectrode assembly 4×4 cm with a ball grid coating. The anode and cathode are switched in alternation resulting in optimal stimulation of the Aδ and C nerve fibers running in the cutis and subcutis. A vibration unit was placed on the electrode to stimulate the Aβ fibers. The following test methods have crystallized out of the quantitative sensory testing that was conducted and have been investigated in greater detail through multiple individual tests.

The following test methods have crystallized out of the Quantitative Sensory Testing that was performed and have been investigated in great detail through multiple individual tests.

### Mechanical Pain Threshold (MPT)

This test was performed using needle stimulus stimulators (pinprick). The threshold was determined in five series of ascending and descending stimulus intensity. The geometric mean of five stimulus intensities above the threshold and five below the threshold is given as the threshold (modified limit value method).

### Pressure Pain Threshold (PPT)

With the help of a pressure algometer (contact area 1 cm²), the threshold for pressure pain above a muscle (see Addendum I) was measured in three series of slowly increasing stimulus intensities (0.5 kg/s, corresponding to approx. 50 kPa/s). The arithmetic mean of these three series is given as the threshold (in kPa).

### Results:

### Mechanical Pain Threshold (MPT)

For N=39 (n=2340), the mechanical pain threshold was calculated at 1, 3 and 4 Hz. The results illustrate the fact that the pain threshold is increased by a factor of 2 to 3 for both of the types of electrodes used. The mechanical pain threshold is between 40 mN and 80 mN for the control area and the placebo. However, this threshold increases to an average of 100-200 mN. The following mean values are reached with each type of electrode:

| Concentric electrode | Spiny electrode |
|---|---|
| 1 Hz: 185 mN | 98 mN |
| 3 Hz: 123 mN | 210 mN |
| 4 Hz: 149 mN | 117 mN |

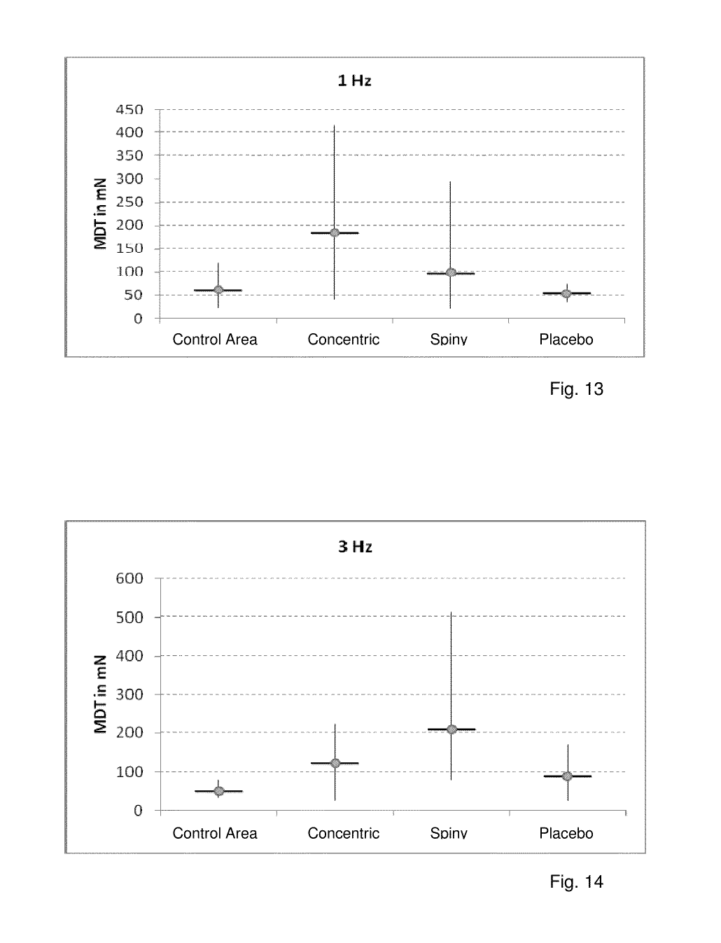

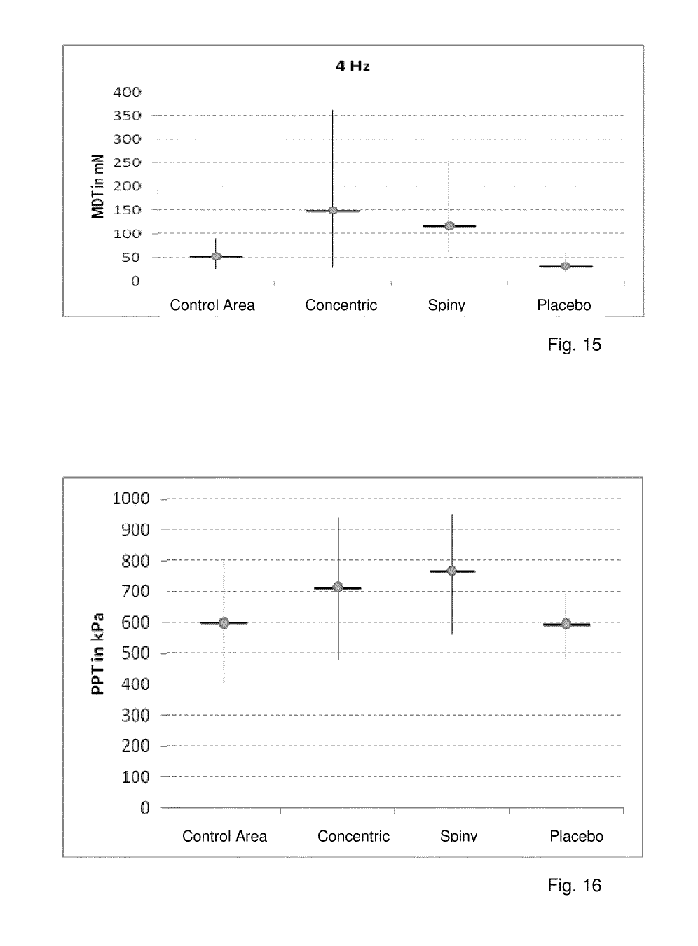

FIGS. 13 to 15 illustrate the results.

### Pressure Pain Threshold (PPT)

For N=22 (n=132) the pressure pain threshold was determined in the control area and the test area after stimulation. Tests were performed with N=11 for the concentric electric and N=11 for the spiny electrode. It was found that after stimulation, a shift in pressure pain threshold by 23.8% (concentric electrode) and 18.4% (spiny electrode) is achieved. FIG. 16 illustrates the results.

### SUMMARY

This test shows that an electrode assembly according to the invention has a significant influenced on the mechanical pain threshold and on the pressure pain threshold.

### Second Example

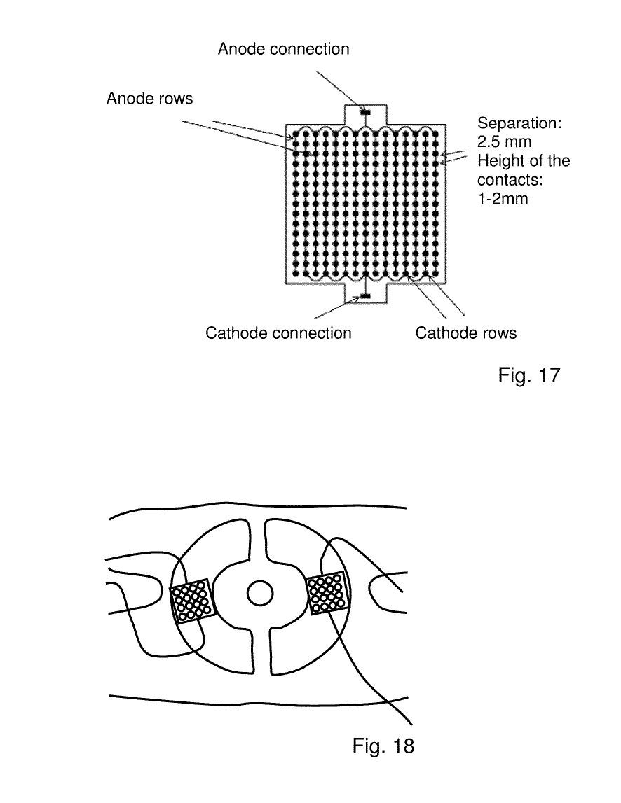

In a second experimental series the function pattern that was developed (bandage with spiny electrode) (see FIGS. 17 and 18) was tested in pressure pain on the knee.

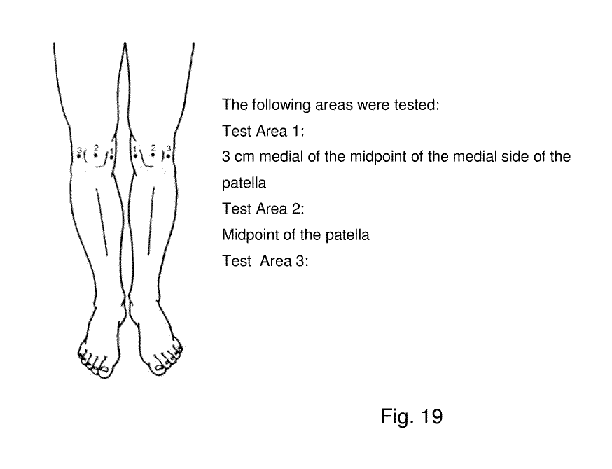

For the test procedure a hand-held algometer (Somedic AB, Sweden) was used. The probe tip (1 cm²) was tested on three predetermined skin points in the peripatellar region (see FIG. 19). The pressure was applied at the rate of 30 kPa/s until the test subject reported that the pressure was perceived as painful.

Six people were tested with the following sequence pattern:
Each person was tested a total of 6 days of three passes each at the Bonn University Clinic (6×3=18 passes)
Days 1 and 2 were performed without stimulation (baseline)
Days 3 and 4 were performed with sham (sham)
Days 5 and 6 were performed with the function pattern (including the electrode assembly)
Each stimulation lasted 15 minutes (effective stimulation pattern: see first example) and was repeated after 45 minutes
The pressure pain threshold (PPT) was tested in the following intervals: 0, 5, 15 and 30 minutes

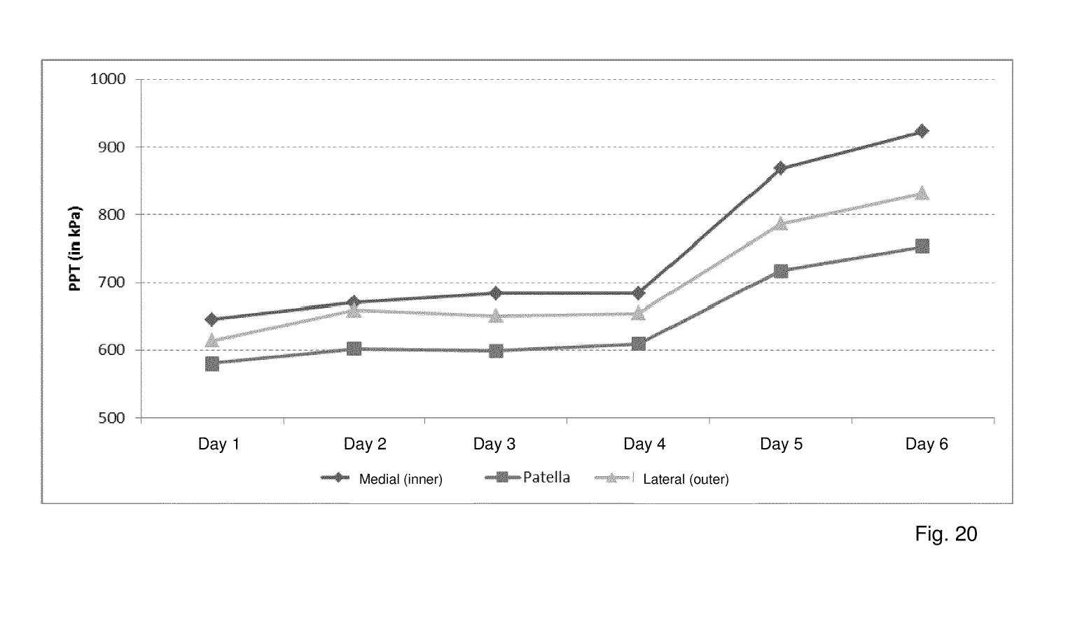

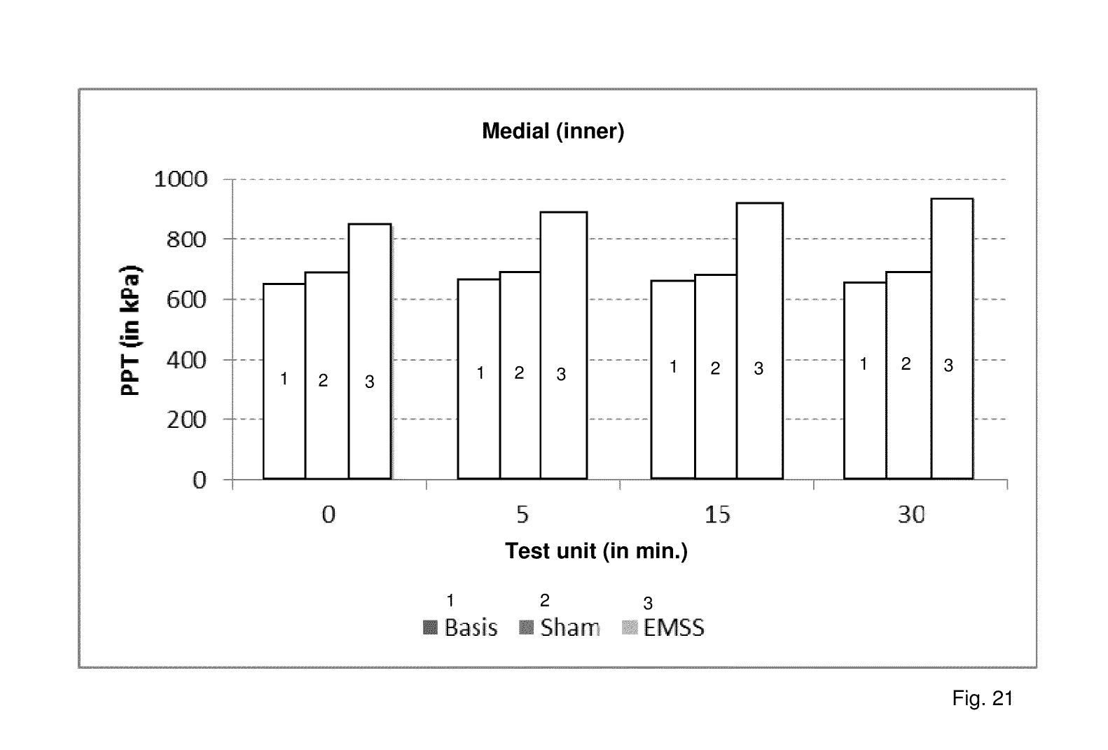

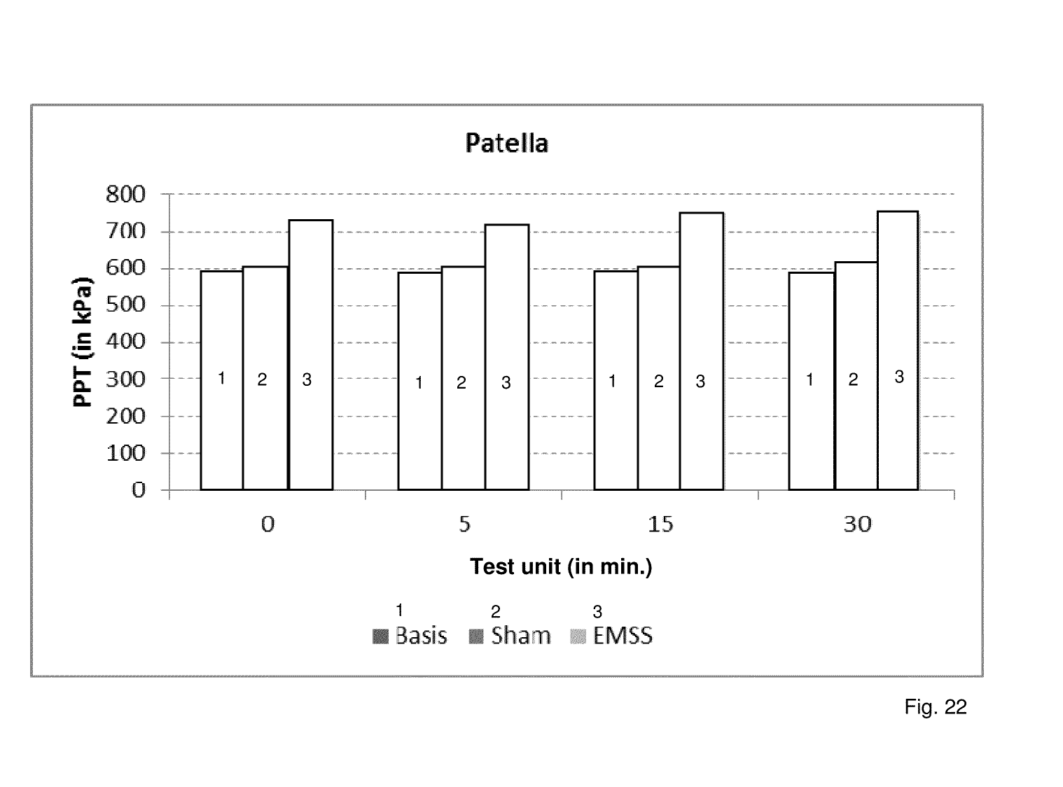

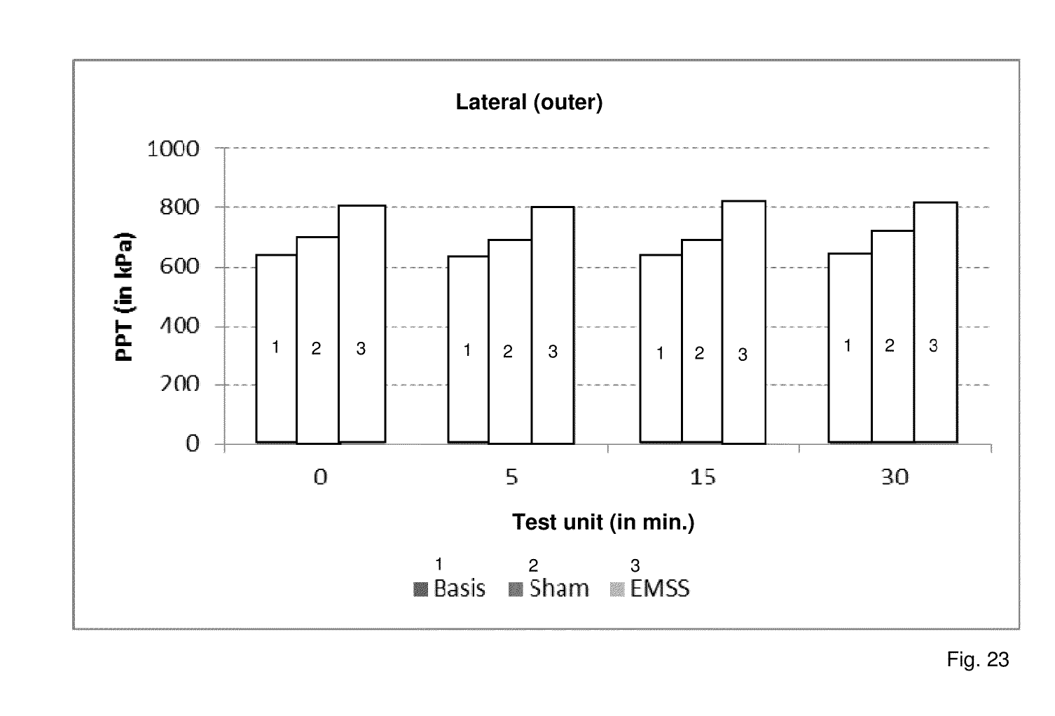

FIGS. 20 to 23 summarize the initial data collected.

The following test results can be summarized:
The pressure pain threshold is reached (at approx. 700 kPa) soonest in test area 2 (patella) in comparison with the lateral side (approx. 800 kPa) and the medial side (approx. 850 kPa).
After stimulation the pain threshold is definitely elevated in comparison with both the baseline and the sham.
The maximum is reached in the pain threshold in the interval at 5-10 minutes after stimulation. The increased pain threshold persists for up to 30 minutes after stimulation.
With repeated stimulation (45-minute cycle), the pain threshold is increased. Although it begins to decline again on day 2, it is elevated in comparison with day 5.

It is found that the function pattern achieves a detectable pain reducing effect with the EMPS that is used.

### Third Example

The electrode assembly is identical to the electrode assembly in the second experimental series (flat electrode assembly in an array). In addition, a vibration motor that would set the electrode assembly in vibration and was connected to the electrode assembly was used. A PicoVibe™ motor 4 mm vibration motor, 8 mm type according to Data Sheet 304-008 from Precision Microdrives™ was used as the vibration motor.

The experimental series with five test subjects showed a further increase in the pain threshold after a stimulation period of 5 minutes in comparison with the first experimental series. The additional vibration thus led to a further reduction in pain and/or pain suppression.

1-38. (canceled)

39. A device for use on the surface of a human or animal body, comprising an electrode assembly, the surface of which can be brought into contact with the surface of the body, a pulse generating unit in electrical communication with the electrode assembly for applying current to the electrode assembly, and a vibration unit physically coupled to the electrode assembly for vibration of the electrode assembly.

40. The device according to claim 39, wherein the electrode assembly comprises a plurality of anodes and a plurality of cathodes, in which the distance between an anode and a neighboring cathode is less than 5 mm.

41. The device according to claim 39, wherein the electrode assembly has concentric electrodes.

42. The device according to claim 39, wherein the pulse generating unit is configured to apply current at a frequency of 0.1 to 20 Hz to the electrode assembly.

43. The device according to claim 39, further comprising measurement and control unit in communication with the pulse generating unit and configured to regulate the current applied to the electrode assembly.

44. The device according to claim 39, wherein the electrode assembly
a) is flexible, and has one or more of
b) gel electrodes and
c) textile electrodes.

45. The device according to claim 39, further comprising a device for supplying energy, said device being connected to the pulse generating unit.

46. The device according to claim 39, wherein the electrode assembly is provided with a coating of a pharmacologically safe class of substances.

47-50. (canceled)

51. The device according to claim 39, wherein the electrode assembly comprises a plurality of electrodes, and at least some of the electrodes each have an area of at least about 1200 mm².

52. The device according to claim 51, wherein the electrodes have a hemispherical surface for contact with the skin.

53. A device for application on skin of a human or animal, comprising:
an electrode assembly, comprising a plurality of electrodes on a flexible carrier structure, for application to the skin;
a current generating unit in electrical communication with the electrode assembly, for providing a stimulus current to the electrode assembly; and
a vibrating unit physically coupled to the electrode assembly, for imparting vibratory motion to the electrode assembly.

54. The device according to claim 53, further comprising a control unit in communication with the current generating unit for regulating the stimulus current provided to the electrode assembly.

55. The device according to claim 54, wherein the control unit is configured to cause the current generating unit to provide a stimulus current of amplitude between 1 mA and 15 mA, in pulses having a pulse period between 100 μs and 500 μs, and a pulse rate between 0.01 Hz and 100 Hz.

56. The device according to claim 55, wherein the control unit is configured to cause the current generating unit to provide an initial stimulus current at a pulse rate between 50 and 100 Hz, and a subsequent stimulus current at a pulse rate between 0.01 Hz and 5 Hz.

57. The device according to claim 54, wherein the control unit is configured to cause the current generating unit to provide a pulsed stimulus current at a frequency of 0.1 to 20 Hz to the electrode assembly.

58. The device according to claim 53, wherein the electrodes comprise gel electrodes.

59. The device according to claim 53, wherein the electrodes comprise printed conductive electrodes on a textile substrate.

60. The device according to claim 53, wherein the electrodes comprise a plurality of anodes and a plurality of cathodes, in which the distance between each of the anodes and a nearest neighboring cathode is less than 5 mm.

61. The device according to claim 60, wherein the distance between each of the anodes and the nearest neighboring cathode is between 1.5 mm and 3.5 mm.

62. The device according to claim 53, wherein the electrode assembly is configured as a ball grid.
